(19) United States
(12) Patent Application Publication
(10) Pub. No.: US 2004/0015133 A1
(43) Pub. Date: Jan. 22, 2004

(54) Epidural apparatus

(76) Inventor: Hussain Karim, Durham (GB)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/296,204

(22) PCT Filed: May 29, 2001
(86) PCT No.: PCT/GB01/02381

(30) Foreign Application Priority Data
May 31, 2000 (GB) 0013039.3
Apr. 4, 2001 (GB) 0108466.4

(51) Int. Cl. A61M 5/178

(52) U.S. Cl. 604/164.07; 604/164.09

(57) ABSTRACT

Epidural apparatus comprises (a) an epidural needle comprising a needle shaft having a tip to allow insertion of a distal end of the needle shaft through the skin of a human or animal body; (b) a sheath comprising a tubular member of plastics material adapted to surround a length of the shaft of the epidural needle but terminate short of the tip thereof, which tubular member, at a distal end thereof is also capable of penetration through the skin following insertion into the skin of the tip of the shaft of the epidural needle and which epidural needle is thereafter removable from the sheath; and (c) a catheter capable of passage through the sheath into the human or animal body.

This invention relates to epidural apparatus comprising an epidural needle and a catheter capable of insertion through the needle into a human or animal body. Such apparatus is used, in many surgical and obstetrical cases, to perform epidural analgesia or anaesthesia.

As shown, for example, in U.S. Pat. No. 5,378,241, conventionally, an epidural apparatus for performing analgesia or anaesthesia comprises a Tuohy needle, the shaft of which has a curved tip which lies away from the axis of the needle. A Luer hub connects the catheter to a bacterial filter and syringe.

In the epidural apparatus, the catheter may be replaced by a spinal needle and various means may be provided for controlling the distance by which the spinal needle protrudes from the epidural needle, thereby controlling the placement of the spinal needle tip within the human or animal body (see EP-A-0872215 and U.S. Pat. No.4,940,458).

When using an epidural apparatus comprising an epidural needle and a catheter, the needle shaft is inserted through the skin, supraspinus ligament, intraspinus ligament, ligamentum flavum and then into the epidural space. The catheter is then threaded through the needle into the epidural space. During adjustment of the length of the catheter within the body and during use of the catheter, it is necessary for the needle shaft to remain within the body.

A problem which arises during the above operation, especially upon withdrawal of the catheter through the epidural needle and during adjustment of the length of the catheter within the body, is that the tip of the needle, especially the curved tip of a Tuohy needle, may cause damage to the catheter and may even transect it. Moreover, such damage would also be likely if the catheter were withdrawn through the needle. In addition, there may also be obstruction to threading the catheter through the needle if the bevel of the Tuohy needle tip is partially obstructed by the ligamentum flavum.

Another problem is that, in order to accommodate the catheter, Tuohy needles need to be particularly wide and such wide needles increase the risk of soft tissue damage, haematoma formation and nerve injury during withdrawal of the needle. Moreover, again because of the large size of the Tuohy needle, which poses a risk of tissue damage in children, there is a limited use of currently available epidural apparatus in paediatric analgesia.

There is also a need to ensure fine control of the position of the shaft of the epidural needle during insertion to avoid the risk of penetration into the subdural space (“dural tap”).

In addition, problems may arise from the use of a Luer hub, employed in conventional epidural apparatus to connect the proximal end of the catheter to a filter. Thus, if over tightened, the Luer hub can obstruct the flow of fluid inside the catheter. On the other hand, if not tightened properly, it may become disconnected, which may, in turn, lead to contamination of the catheter and possible infection.

I have addressed the above problems and have developed an epidural apparatus which at least minimises or eliminates at least some of the above problems.

According to one aspect, the invention provides epidural apparatus comprising

(a) an epidural needle comprising a needle shaft having a tip to allow insertion of a distal end of the shaft through the skin of a human or animal body;

(b) a sheath comprising a tubular member of plastics material adapted to surround a length of the shaft of the epidural needle but terminate short of the tip thereof,

which tubular member, at a distal end thereof, is also capable of penetration through the skin following insertion into the skin of the tip of the shaft of the epidural needle and which epidural needle is thereafter removable from the sheath; and

(c) a catheter capable of passage through the sheath into the human or animal body;

whereby after arranging the sheath around the epidural needle and inserting the tip of the needle shaft and then a distal end region of each of the needle shaft and the sheath into the human or animal body, the epidural needle is removable from the sheath and the catheter is capable of insertion into the human or animal body through the sheath, thereby avoiding damage by the epidural needle of the catheter during passage through the tip thereof on insertion, adjustment of the length of the catheter within the body or removal of the catheter from the body.

A preferred sheath additionally comprises a generally cylindrical hub member secured to an axial end of the tubular member and coaxial therewith, the hub member having a hollow bore so as to allow access of the needle shaft therethrough and through the tubular member.

Likewise, a preferred epidural needle additionally comprises a generally cylindrical hub member secured to an axial end of the needle shaft and coaxial therewith, the hub member having a hollow bore so as to allow access therethrough and through the needle shaft.

Preferably, the epidural-apparatus additionally comprises means adapted to secure the epidural needle and sheath to, or remove the epidural needle and sheath from, one another. More preferably, the means are additionally adapted to provide controlled relative axial movement of the epidural needle and sheath, one relative to the other.

An especially preferred means for removably securing together the epidural needle and the sheath are

(1) an internally screw threaded surface defining a bore of a cylindrical collar coaxially surrounding and fixed relative to one of the shaft of the epidural needle and the tubular member of the sheath at a respective proximal end region thereof; and

(2) an externally screw threaded surface of a hub member axially aligned with and fixed relative to the other of the shaft of the epidural needle and the tubular member of the sheath at a respective proximal end region thereof, which externally screw threaded surface is cooperable with the internally screw threaded surface of the collar to allow screw threaded engagement of the hub member within the bore of the collar.

For example, the means for removably securing together the needle and the sheath may be

(1) an internally screw threaded surface defining a bore of a cylindrical collar surrounding and fixed relative to the shaft of the epidural needle and

(2) an externally screw threaded surface of a hub member of the sheath at a proximal axial end thereof and cooperable with the internally screw threaded surface.

In a preferred epidural apparatus, at least one of the epidural needle and sheath has, surrounding and fixed relative thereto, a respective disc-like calibration member provided with marking so as to allow an indication of the extent of relative revolution one to another and thereby, from the pitch of the screw threads, allow calibration of the extent of relative axial movement.

More preferably, the disc-like calibration is provided on the tubular or hub member of the sheath.

Especially preferably, the tubular member is provided with marks, typically in cm, to provide a visual guide as to the depth of penetration of the shaft into the body.

The sheath may be an integral unit of plastics material, the tubular member projecting axially from an axial end of the hub member. Preferably, the tubular member is of flexible plastics material.

Alternatively, the tubular member and the hub member of the sheath may be separate components, the tubular member fitting tightly at a proximal end thereof within the distal end of a bore of the hub member. Again, preferably, the tubular member is then preferably of flexible plastics material. However, the hub member is then preferably of rigid plastics material. Preferably, the bore of the hub member of the sheath has a diameter, at a distal end thereof, essentially equal to the internal diameter of the tubular member and, at a proximal end thereof, a wider diameter.

In addition to the shaft, the epidural needle may additionally comprises a generally cylindrical hub member secured to an axial end of the needle shaft and coaxial therewith, the hub member having a generally hollow bore so as to allow access through the epidural needle. In such a needle, the bore of the hub member preferably has a diameter, at a distal end thereof, essentially equal to the internal diameter of the needle shaft and, at a proximal end thereof, a diameter sufficiently wide to receive a distal end region of a syringe.

The collar surrounding a proximal end of the needle shaft may project axially from a distal end of the hub member.

In a preferred epidural apparatus embodying the invention, each of a hub member of the epidural needle and a tubular or hub member of the sheath carries in fixed relation with and coaxially with the epidural needle and sheath respectively, a respective disc-like core element having a toothed outer circumferential surface and the epidural apparatus additionally comprises an annular disc-like outer element having a marked outer circumferential surface and a toothed inner circumferential surface cooperable, on axial movement thereof along one of the epidural needle and the sheath, with either of the respective core elements upon engagement therewith, so as to provide the said disc-like calibration member on one of the sheath and epidural needle as desired.

In an especially preferred epidural apparatus the epidural needle has a body part which is a one-piece moulding of plastics material having a generally hollow bore passing axially therethrough, the body part comprising, (1) at a proximal end region, a hub part capable of receiving within the bore the hub of a syringe and capable of being held externally by the user, (2) at a distal end region, a cylindrical collar projecting axially away from a distal end of the hub member and having an internally screw threaded bore adapted to receive an externally screw threaded sheath and, (3) in a medial axial region of the body part, a disc-like core element provided by a portion of greater radial extent than the hub member and having a toothed outer circumferential surface adapted to receive an annular disc-like outer element having a correspondingly toothed inner circumferential surface and a marked outer surface.

An epidural apparatus embodying the invention may additionally include

(1) a stylet comprising an essentially cylindrical solid rod adapted to pass into the epidural needle up to the tip of the needle shaft and thereby block the distal end of the needle shaft during insertion thereof into a human or animal body, thereby preventing egress of matter into the needle shaft; and

(2) means adapted to removably secure the epidural needle and stylet to one another.

The means for removably securing together the stylet and the epidural needle may be

(1) an internally screw threaded surface defining a bore of a cylindrical collar coaxially surrounding and fixed relative to one of the stylet and the epidural needle at a respective proximal axial end region thereof; and

(2) an externally screw threaded surface of a hub member axially aligned with and fixed relative to the other of the stylet and the epidural needle, which externally screw threaded surface is cooperable with the internally screw threaded surface of the collar to allow screw threaded engagement of the hub member with the bore of the collar.

The means for removably securing together the stylet and the epidural needle may be

(1) an internally screw threaded surface defining a bore of a cylindrical collar surrounding and fixed relative to a proximal end region of the stylet and

(2) an externally screw threaded surface of a hub member of the epidural needle at a proximal end region thereof and cooperable with the internally screw threaded surface of the collar.

In respective different embodiments of the invention, an axial end of the catheter is either abused with a filter or is fused with a hub provided on a filter.

According to another aspect, the invention provides a method of locating a catheter within an epidural space of a human or animal, which method comprises

(1) providing (a) an epidural needle comprising a needle shaft having a tip to allow insertion of a distal end of the needle shaft through the skin of a human or animal body and (b) a sheath comprising a tubular member of plastics material adapted to surround a length of the shaft of the epidural needle but terminate short of the tip thereof and capable of penetration through the skin following insertion into the skin;

(2) inserting into the skin a distal end region of each of the shaft of the epidural needle and the tubular member of the sheath until the tip of the needle shaft reaches the epidural space;

(3) thereafter removing the epidural needle from the sheath to leave only the distal end region of the tubular member of the sheath within the body; and thereafter

(4) inserting the catheter into the epidural space by passage thereof through the sheath.

In a preferred method, the tubular member is of flexible plastics material, means are provided for controlling relative axial movement of the epidural needle and sheath one relative to the other, and step (2) is carried out by

(i) arranging the tubular member of sheath around the shaft of the epidural needle at a position where the distal end of the tubular member terminates short of the tip of the shaft;

(ii) inserting the tip of the shaft of the epidural needle and a distal end region of the tubular member into the body to a position no further than the ligamentum flavum;

(iii) advancing the distal end of the tubular member axially relative to the shaft until resistance to the movement is sensed;

(iv) advancing the tip of the needle shaft through a predetermined minimal axial extent relative to the distal end of the tubular member;

(v) again advancing the distal end of the tubular member axially forward of the needle shaft and sensing the resistance thereto;

(vi) repeating steps (iv) and (v) until no resistance is sensed, thus locating the tip of the needle shaft in the epidural space; and

(vii) further advancing the flexible tubular member axially forward of the needle shaft into the epidural space.

In an alternative method, one or both of steps (iii) and (v) may be preceded by a step of inserting a low resistance syringe into the sheath and testing whether forward operation of the syringe meets with any resistance. When no such resistance is met, the epidural space has been reached. Subsequent step (iii) or (v) then confirms the result of such a test.

Especially preferred embodiments of the invention will now be described in more detail with reference to the accompanying drawings which are roughly on a scale of 2:1 and in which:

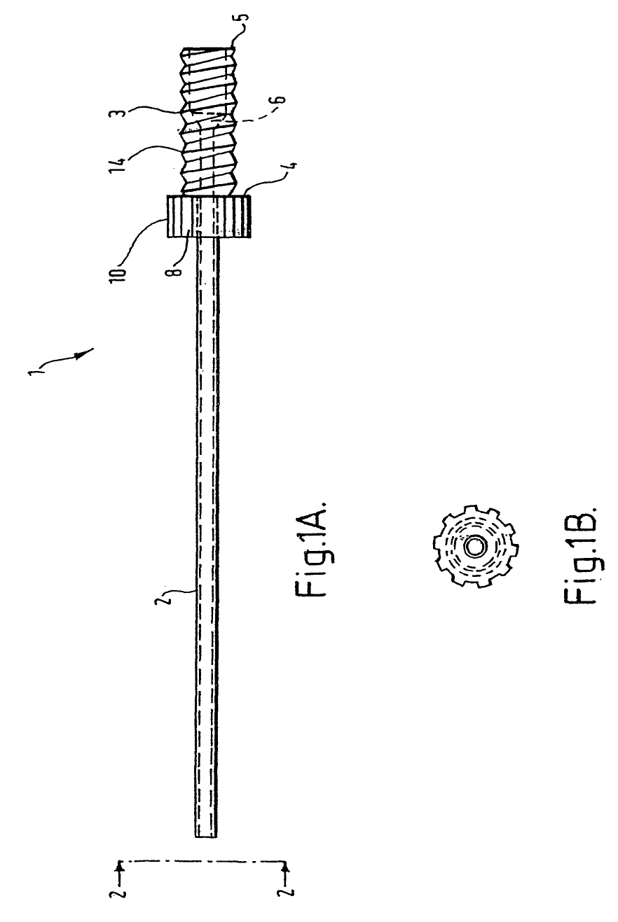

FIG. 1 is a side view of a sheath of an epidural apparatus embodying the invention and FIG. 1B is a radial view from a distal axial end in the direction of arrows A of FIG. 1A;

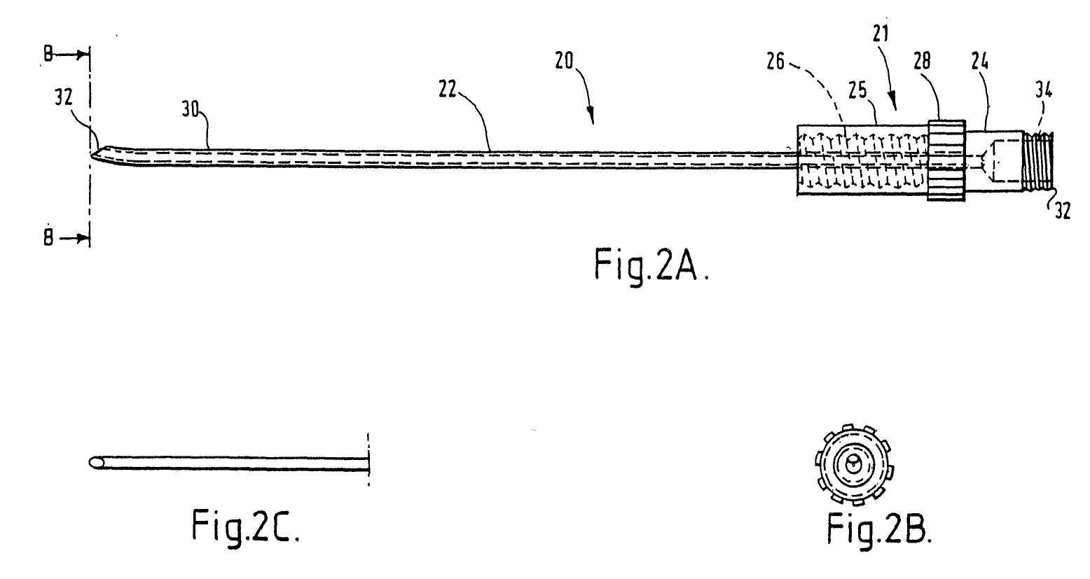

FIG. 2A is a side view of an epidural needle engageable with the sheath shown in FIGS. 1A-1B, FIG. 2B is a radial view from a distal axial end thereof in the direction of arrows B of FIG. 2A, and FIG. 2C is a plan (posterior) view (from below) of a distal end region of the needle shaft of FIG. 2A;

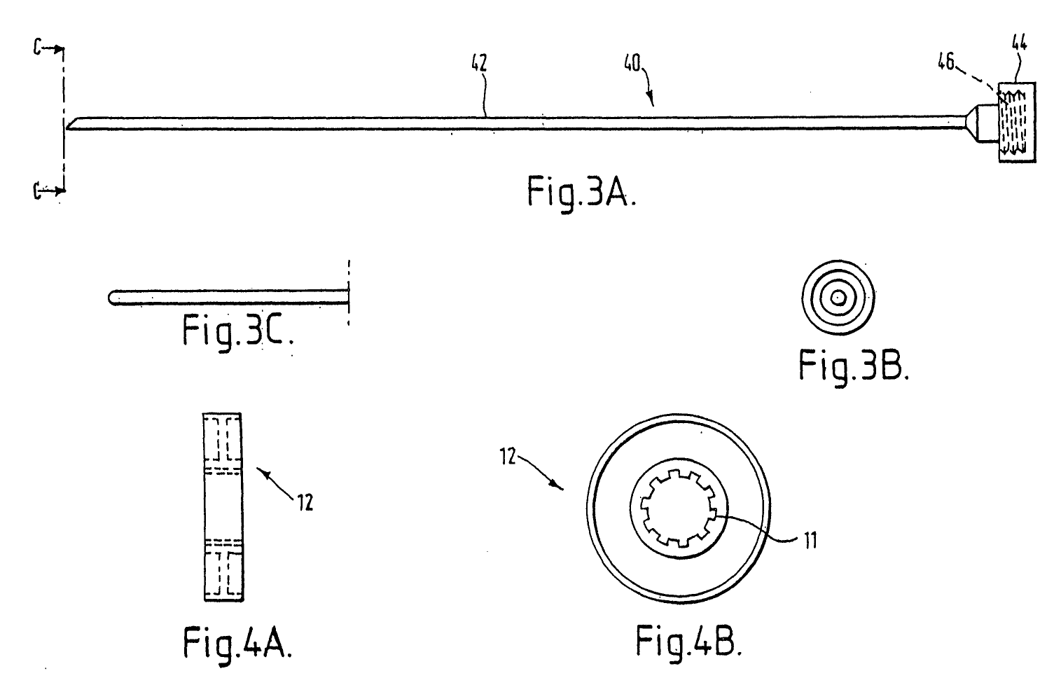

FIG. 3A is a side view of a stylet engageable with an epidural needle shown in FIGS. 2A-2C, FIG. 3B is a radial view from a distal axial end thereof in the direction of arrows C of FIG. 3A and FIG. 3C is a plan (posterior) view (from below) of a distal end region of the stylet of FIG. 3A;

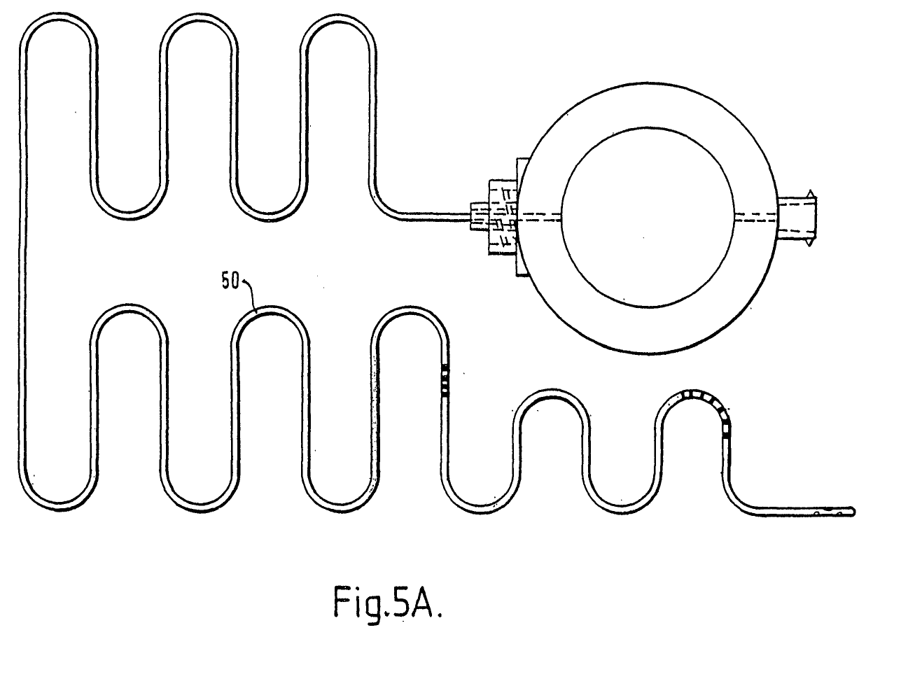

FIG. 4A is a side view of an annular outer disc element designed to accommodate an inner disc element located on the tubular member of the sheath shown in FIGS. 1A-1B and FIG. 4B is a radial view from either axial end of the annular outer disc element of FIG. 4A; and

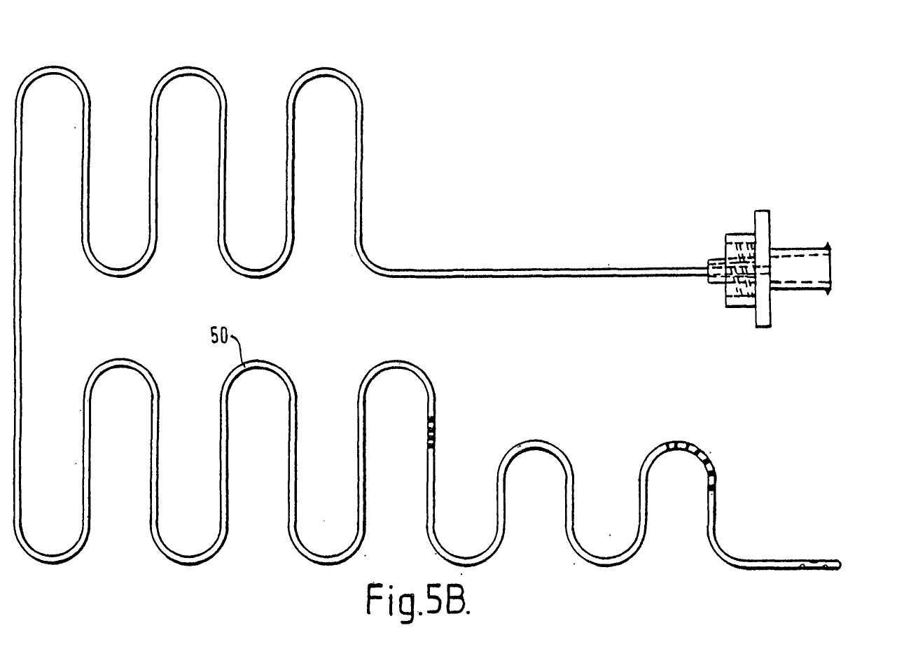

FIG. 5A is a side view of a catheter engageable with a sheath as shown in FIGS. 1A-1C, fused to a filter while FIG. 5B is a side view of such a catheter fused to. a hub adapted to receive the filter.

Referring firstly to FIGS. 1A-C, these show an epidural sheath 1, comprising a tubular member 2 of flexible plastics material, a proximal end region of which fits into a distal end region of a hub member 3 of rigid plastics material coaxial with the tubular member 2. The tubular member 2 may be provided with markings along its length, typically in centimetres. The hub member 3 has an internal bore which, at a distal end 4 thereof has a diameter essentially equal to the internal diameter of the tubular member 2 and at a proximal end 5 thereof has a wider diameter. A medial region of the bore has a frustoconical section 6 diverging from the region of narrower to the region of wider diameter. The bore so constructed allows the sheath to receive (i) a syringe to allow fluid injection or (ii) an adapter having a narrower axial passage through it to assist in threading the catheter through the sheath or (iii) a hub on a filter after removal of the sheath. To this end, the sheath may be provided at its proximal end with a radially outwardly projecting circumferential flange threadedly engageable within an internal screw thread on the hub of a filter or a hub provided at the proximal end of the catheter.

One particular embodiment of sheath is provided with means, for example, a line or region of weakening, thus rendering it easily breakable and thereby separable from the catheter for disposal.

A distal axial end of the hub member 3 is of increased external diameter such as to define a disc-like core element 8 or, in an alternative embodiment, secured to and circumferentially surrounding the tubular member 2 is a disc-like core element 8 axially adjacent the distal end of the hub member 3. In either case, the disc-like core element 8 has a toothed outer surface 10 capable of insertion into a correspondingly toothed inner surface 11 of a marked annular disc-like outer element 12 shown in FIG. 4. This outer element 12 serves to allow a user to control accurately the axial movement of the sheath and epidural needle relative to one another, as later described.

Finally, the hub member 3 of the sheath 1 has an external screw thread 14 to allow engagement within a collar 25 of an epidural needle 20 as described below with reference to FIGS. 2A-2C.

As to the dimensions of the sheath 1, typically, the tubular member 2 is from 25 to 150 mm in length, more typically 50-100 mm, especially 50-75, for example, say 50 mm for paediatric use, 75 mm for a standard adult and 100 mm for an obese adult patient, from 0.85 to 1.65 mm in inside diameter, from 1.0 to 1.8 mm outside diameter, and from 0.075 to 0.2 mm in thickness, more typically 0.075 to 0.15 mm, especially 0.075 mm. Typically the hub member 3 is from 15 to 20 mm in length, more typically 17 to 19 mm, especially 19 mm, of which 3 to 5 mm defines the disc-like core element 8 at the distal end and the remainder of which is provided with a screw thread, typically of a pitch of from 2 to 4 mm. The outer diameter of the core element 8 is typically 5-10 mm, especially 18 mm with, say 2 mm deep, axially extending recesses defining teeth. The outer diameter of screw threaded part is typically 5 to 6 mm with a screw depth of about 1 mm. Typically the diameter of the internal bore passing through the hub member 3 is, at a distal end region of the hub member 3 defining the core element 8, such as to allow a press fit into the bore of the flexible tubular member 2, while at a distal end of the screw threaded part of the hub member 3, the diameter is the same as the internal diameter of the tubular member 2. At a medial axial region of the screw threaded part, a frusto-conical length of the bore, typically 1 to 3 mm, especially 1.5 mm diverges towards a proximal end up to a diameter of from 3 to 5 mm, typically 4 mm, in a proximal end region having a length approximately half that of the screw threaded portion of the hub member 3, typically 3 to 8 mm, especially 6.5 mm in length.

In particular, the internal diameter of the tubular member 3 of the sheath 1 should be sufficiently wide to accommodate the epidural needle.

The above sheath 1 is an example of a novel sheath comprising a tubular member of plastics material adapted to surround the length of a shaft of an epidural needle and a hub member screw threadedly engagable with the epidural needle to allow relative rotation and consequent relative axial movement so as to allow fine axial adjustment of the position of the shaft.

An epidural apparatus embodying the invention also comprises, as shown in FIGS. 2A-2C, an epidural needle, generally indicated as 20, comprising a needle shaft 22 of metal projecting axially outwardly from a distal end of a body part, generally indicated as 21, and including a hub 24 to which the needle shaft 22 is secured. Also projecting outwardly from the distal end of the hub 24 is a collar 25 of the body part 21, integral with the hub 24, coaxial with the needle shaft 22 and having an internally screw threaded surface defining a bore 26 adapted to receive the screw threadedly engageable hub member 3 of the sheath 1. As can be seen, the respective axial lengths of the collar 25 of the epidural needle 20 and hub member 3 of the sheath 1 may be the same.

Axially adjacent to a proximal end of the collar 25 and secured to or integral with a distal end region of the hub 24 is an annular disc-like element 28 of the body part 21 circumferentially surrounding the hub 24 and having a toothed outer surface thus providing a disc-like core element additional or alternative to the disc-like core element 8 on the sheath 1 for cooperation with a toothed inner surface of a marked annular disc-like outer element 12, as shown in FIG. 4. The marked outer element 12 is thereby attachable to the epidural needle 20 so allowing the user to select an alternative or additional means of controlling accurately the axial movement of the sheath and epidural needle relative to one another, as later described.

More particularly, as desired by the user, a marked annular disc-like outer element 12 can be passed axially over the assembly of sheath 1 and epidural needle 20 from either axial end until it is brought into contact with a selected one of the disc-like core elements 8 and 28 on the sheath 1 and epidural needle 20 respectively. The respective teeth in the core and outer elements may then be aligned with one another so as to allow interengagement. Preferably the interengagement is achieved by a press or snap fit. Alternatively, two annular disc-like outer elements 12 can likewise be engaged, one with each of the respective core elements 8 and 28.

Since the pitch of the screw threads of collar 25 of epidural needle 20 and hub member 3 of sheath 1 are known, the extent of revolution of an annular disc-like element 12, for example, attached to the tubular member 2 of the sheath 1 during screw threading of the hub member 3 within collar 25 can be calibrated against the axial displacement caused by such revolution For example, where the pitch of the screw thread is 2 mm, rotation of the sheath through 360° will advance or retract the tubular member relative to the sheath through an axial length of 2 mm, while if the pitch is 4 mm a rotation of only 180° will provide an axial movement of 2 mm. This allows an accurate assessment of the extent of relative axial displacement, even down to 1 mm or less.

Preferably, the hub 24, disc-like core element 28 and collar 25 of the epidural needle 20 are provided by a one-piece moulding of plastics material.

The hub 24 of the epidural needle 20 also has an internal bore which, at a distal end thereof has a diameter essentially equal to the internal diameter of the needle shaft 22 and, at a proximal end 32 thereof, has a diameter sufficiently wide to receive a distal end region of a syringe for injection of fluid, for example, air, as when providing one method of accurately locating the position of the tip 32 of the needle, as later described.

The proximal end 32 of the epidural needle 20 also has an external screw thread 34 for connection to a stylet 40 shown in FIGS. 3A-3C.

As to the dimensions of the epidural needle 20, typically the needle shaft 22 is from 50 to 100 mm in length, more typically 65-85 mm, for example, say 65 mm for paediatric use, 85 mm for a standard adult and 110 mm for an obese adult patient, from 0.5 to 1.35 mm in inside diameter, from 0.7 to 1.55 mm in outside diameter and from 0.5 to 0.2 mm, more typically from 0.05 to 0.15 mm, in thickness. Typically, the body part 21 is from 15 to 35 mm, more typically from 19 to 30 mm, in length, of which 10 to 20 mm defines the collar 25 (which collar 25 surrounds from 10 to 20 mm of the needle shaft 22) at the distal end of the hub member 24, from 3 to 5 mm of which defines a disc-like element 28 axially adjacent to the collar and the remaining length of hub member 24 provides a proximal end region of the hub member 24. The outer diameter of the collar 25 is typically 4-8 mm, more typically 6.5 mm, while the outer diameter of the core element 28 is typically 5-10 mm, especially 8 mm, with, say 2 mm deep, axially extending recesses defining teeth.

The outer diameter of the remaining proximal end region of the hub member 24 is preferably from 3 to 4 mm, with a most proximal axial part having an external screw thread of about 2.5 to 4 mm in length for receipt of a screw threaded collar 44 of the shaft. The diameter of the bore of the collar 25 corresponds to the external diameter of the screw threaded hub member 25 of the sheath 1. The diameter of the internal bore passing through the hub member 24 is, at a medial axial region (of about 3-4 mm in length) defining the core element 28, such as to allow a press fit into the bore of a proximal end of the metal needle shaft 22, while for an axial length (of about 1 to 2.5 mm) adjacent to the core element 28, the diameter of the bore is the same as the internal diameter of the needle shaft 22. Towards a distal end of the hub 24, a frusto-conical length of the bore, typically 1-3 mm especially 2.5 mm, diverges towards a proximal end region up to a diameter of from 3.5 to 5, especially 4 mm, in a proximal end region of axial length, typically from 5 to 12 mm, more typically 6-10 mm.

The above needle 20 is an example of a novel epidural needle which differs from known, so-called Tuohy, needles in that the needle 20 has

a) a collar which is generally axially central of the needle and screw threadedly engagable with the sheath to permit its fine advancement;

b) a reduced thickness, allowing a reduction in damage to the patient during withdrawal of the needle; and

c) a tip having a cross-section less than that of the same guage of Tuohy needle, thus decreasing the incidence of dural tap (eg. for a 16-guage needle, the tip diameter is reduced for 3 mm to 2 mm); this is particularly significant given that the diameter of the epidural space in the thoracic region is 3-5 mm and 5-7 mm in the lumbar region.

As can be seen from FIGS. 3A-3C, epidural apparatus embodying the invention also comprises a stylet 40, which comprises an essentially cylindrical solid rod 42 adapted to pass into the epidural needle 20 up to the tip 32 of the needle shaft 22 and thereby block the distal end 30 of the needle shaft 22 during insertion thereof into a human or animal body, thereby preventing egress of matter into the needle shaft.

At a proximal end of the stylet, a screw threaded collar 44 is secured thereto. This has an internally screw threaded bore 46 cooperable with the externally screw threaded surface 32 of the epidural needle 20 to allow the stylet 40 to be removably engageable with the epidural needle 20.

The epidural apparatus additionally includes a catheter 50 which may be fused directly to a filter (see FIG. 5A) or fused to a hub adapted to receive the filter (see FIG. 5B). Such a catheter is novel.

Thus, a preferred embodiment of the invention is a ‘three-in-one’ epidural set in which each of the sheath, needle and catheter are newly designed to achieve the advantages elucidated more fully below.

Typically, the catheter is from 600-800 mm (excluding hub or filter) in length, from 0.7-1.55 mm in outside diameter, from 0.60-1.45 mm in inside diameter and 0.05 mm in thickness.

As previously mentioned, the purpose of the needle 20 is to provide access for the catheter 50 into a human or animal body and, in particular, into the epidural space. To this end, the needle shaft 22 has a distal end region 30 having a needle tip 32, a side view of which is shown in FIG. 2A and a ventral plan view (from above) of which is shown in FIG. 2C. As can be seen, the needle tip 32 is curved at a 20° angle relative to a horizontal plane and has an internal diameter, typically of 2 mm.

An epidural apparatus embodying the invention allows both accurate location of the needle tip 32 (and therefore the catheter 50) in the epidural space, thus minimising the risk of epidural tap, while at the same time avoiding the risk that the catheter 50 might be damaged by the needle tip 32 while either introducing into or removing it from a human or animal body or while adjusting the position of the catheter within the body.

Thus, the following steps may be performed in order to fit a catheter 50 into a human or animal body:

(i) the sheath 1 and epidural needle 20 are held, for example, by their respective hub members 3 and 24 and brought together to allow screw threaded engagement of the hub member 3 of sheath 1 into the collar 25 of the epidural needle 20. Screw threading is continued until the tubular member 2 of the sheath 1 terminates short of the distal end of the needle shaft 22 of the epidural needle 20; ideally, the total length of the sheath 1 is such that, when the proximal end of sheath is located in an axially central region of the collar 25, the distal end of the tubular member 2 of the sheath terminates just short of the distal end of the needle shaft 22.

(ii) Distal end regions of shaft 22 of the epidural needle 20 and tubular member 2 of the sheath 1 are then inserted into the body.

(iii) Holding the sheath 1, the epidural needle 20 is then rotated clockwise to advance it through a small distance, e.g. 1 mm, so that it penetrates slightly further into the body.

(iv) Holding the epidural needle 20, the sheath 1 is rotated anticlockwise to move it distally until the tubular member 2 of the sheath 1 extends up to or beyond the tip 32 of the epidural needle 20.

(v) If the tip 32 of the epidural needle 20 is still in the ligamentum flavum, resistance to further rotation of the sheath is met.

(vi) In such a case holding the sheath 1, the epidural needle 20 is screwed clockwise so that the tip 32 of the needle shaft 22 emerges from the sheath 21 but only to an extent of about 1-2 mm, as desired, in the hope that the tip 32 of the epidural needle has reached the epidural space without penetration of the dura matter.

(vii) Again, holding the epidural needle 20 and rotating the sheath 1 anticlockwise, if at this stage, no resistance is met, then the sheath has reached the epidural space.

(viii) The sheath 1 is then further rotated anticlockwise to provide a sufficient length of the sheath within the epidural space for location of the catheter 50.

(ix) With the sheath 1 in place, the entire epidural needle 20 is then removed and disposed of and the catheter 50 is threaded into the sheath 1 and into the epidural space with no resistance.

(x) The sheath 1 is then removed and engaged with a hub on the filter.

Bearing in mind that the width of the epidural space is only 3-5 mm for the thoracic region and 5-7 mm for the lumbar region, by this method, especially by attempting to advance the epidural needle mm by mm until subsequent anticlockwise rotation of the sheath meets no resistance, it is possible to ensure that the tip of the epidural needle does not puncture the meninges layer and enter into the subdural space.

As an alternative to using the tip of the sheath for assessment of resistance, it is possible to attach a syringe to the internal bore of the proximal end of the hub member 3 of the sheath 1 and check whether operation of the syringe meets any resistance.

In the manner described above, the problem of possible damage of the catheter upon adjustment of its position in the body or withdrawal through the epidural needle by the curved tip of the needle is avoided. Likewise, there is considerably less likelihood of any obstruction of the end of the tubular member of the sheath as compared with the corresponding difficulties arising from the bevel of the Tuohy needle tip as previously explained.

Moreover, the use of a sheath allows a much smaller diameter shaft 22 of the epidural needle to be used whilst retaining the standard catheter diameter thereby allowing application of the epidural apparatus in paediatric anaesthesia.

Thus, typical dimensions of component parts of sets of epidural apparatus embodying the invention are now given and compared with those of sets of known apparatus, each referred to as a “previous set”. In each of the Tables below, “O.D.” refers to the outside diameter “I.D.” refers to the inside diameter and all dimensions are in mm.

### Calibrated Sheathed Epidural Sets

1—16 Gauge

| | O.D. | I.D. | Thickness | Length | |
|---|---|---|---|---|---|
| Sheath | 1.60 | 1.45 | 0.075 | 75 | mm (tubular member only, |
| | | | | | excluding hub member), |
| | | | | 94 | mm (total length including |
| | | | | | hub member) |
| Calibrated | 1.30 | 1.10 | 0.10 | 85 | mm (needle shaft only, |
| Epidural | | | | | excluding hub member), |
| Needle | | | | 114 | mm (total length including |
| | | | | | hub member) |
| Epidural | 1.30 | 1.20 | 0.05 | 800 | mm, excluding filter. |
| Catheter | | | | 850 | mm, including filter. |

Previous Set:.

| | | | | | |
|---|---|---|---|---|---|
| Tuohy | 1.60 | 1.15 | 0.225 | 80 | mm, (needle shaft only, |
| Needle | | | | | excluding hub member) |
| | | | | 110 | mm (total length |
| | | | | | including hub member) |
| Epidural | 1.00 | 0.90 | 0.05 | 900 | mm, excluding hub |
| Catheter | | | | | |

As can be seen from the above Table, for a “16 guage” apparatus, in which the widest outer diameter of the assembly for insertion into a human or animal body is 1.60 mm, since the presence of the Tuohy needle is no longer necessary for guiding the catheter into the body, a larger inner diameter aperture (provided by the inner diameter of the sheath) is available for passage of the catheter. This allows the epidural catheter to have inner and outer diameters each 30% larger than in the corresponding known apparatus, so decreasing the resistance inside the catheter and enabling an increase in the flow rate of infusion of about 400%.

In addition, since the epidural needle no longer needs to accommodate the catheter, its inner and outer diameters at the needle tip can be reduced, thus reducing the risk of soft tissue damage, haematoma formation and nerve injury, especially during withdrawal of the needle.

2—18 Gauge:

| | O.D. | I.D. | Thickness | Length | |
|---|---|---|---|---|---|
| Sheath | 1.30 | 1.15 | 0.075 | 75 | mm (tubular member, |
| | | | | | excluding hub member), |
| | | | | 94 | mm (total length including |
| | | | | | hub member) |
| Calibrated | 1.00 | 0.80 | 0.10 | 85 | mm (needle shaft only, |
| Epidural | | | | | excluding hub member), |
| Needle | | | | 114 | mm (total length including |
| | | | | | hub member) |
| Epidural | 1.00 | 0.90 | 0.05 | 800 | mm, excluding hub. |
| Catheter | | | | 850 | mm, including filter. |

Previous Set:

| | | | | | |
|---|---|---|---|---|---|
| Tuohy | 1.30 | 1.00 | 0.15 | 80 | mm, (needle shaft only, |
| Needle | | | | | excluding hub member), |
| | | | | 110 | mm (total length |
| | | | | | including hub member) |
| Epidural | 0.80 | 0.70 | 0.05 | 900, | excluding hub. |
| Catheter | | | | | |

As in the case of the 16-guage set, the 20% larger diameter catheter which can be used enables an increase in flow rate of infusion, in this case of 260%.

3—14 Gauge

| | O.D. | I.D. | Thickness | Length | |
|---|---|---|---|---|---|
| Sheath | 1.8 | 1.65 | 0.075 | 75 | mm (tubular member only, |
| | | | | | excluding hub member), |
| | | | | 94 | mm (total length including |
| | | | | | hub member) |
| Calibrated | 1.55 | 1.35 | 0.10 | 85 | mm (needle shaft only, |
| Epidural | | | | | excluding hub member), |
| Needle | | | | 114 | mm (total length including |
| | | | | | hub member) |
| Epidural | 1.55 | 1.45 | 0.05 | 800 | mm, excluding filter |
| Catheter | | | | 850 | mm, including filter |

4—15 Gauge:

| | O.D. | I.D. | Thickness | Length | |
|---|---|---|---|---|---|
| Sheath | 1.70 | 1.55 | 0.075 | 75 | mm (tubular member |
| | | | | | excluding hub member), |
| | | | | 94 | mm (total length including |
| | | | | | hub member) |
| Calibrated | 1.40 | 1.20 | 0.10 | 85 | mm (needle shaft only, |
| Epidural | | | | | excluding hub member), |
| Needle | | | | 114 | mm (total length including |
| | | | | | hub member) |
| Epidural | 1.40 | 1.3 | 0.05 | 800 | mm, excluding filter |
| Catheter | | | | 850 | mm, including filter |

5—17 Gauge:

| | O.D. | I.D. | Thickness | Length | |
|---|---|---|---|---|---|
| Sheath | 1.50 | 1.35 | 0.075 | 75 | mm (tubular member only, |
| | | | | | excluding hub member), |
| | | | | 94 | mm (total length including |
| | | | | | hub member) |
| Calibrated | 1.20 | 1.00 | 0.10 | 85 | mm (needle shaft only, |
| Epidural | | | | | excluding hub member), |
| Needle | | | | 114 | mm (total length including |
| | | | | | hub member) |
| Epidural | 1.20 | 1.10 | 0.05 | 800 | mm, excluding filter |
| Catheter | | | | 850 | mm, including filter |

6—19 Gauge:

| | O.D. | I.D. | Thickness | Length | |
|---|---|---|---|---|---|
| Sheath | 1.23 | 1.08 | 0.075 | 75 | mm (tubular member only, |
| | | | | | excluding hub member), |
| | | | | 94 | mm (total length including |
| | | | | | hub member) |
| Calibrated | 0.93 | 0.73 | 0.10 | 85 | mm (needle shaft only, |
| Epidural | | | | | excluding hub member), |
| Needle | | | | 114 | mm (total length including |
| | | | | | hub member) |
| Epidural | 0.93 | 0.83 | 0.05 | 800 | mm, excluding filter |
| Catheter | | | | 850 | mm, including filter |

### Paediatric Sets

7—20 Gauge:

| | O.D. | I.D. | Thickness | Length | |
|---|---|---|---|---|---|
| Sheath | 1.15 | 1.00 | 0.075 | 55 | mm (tubular member, |
| | | | | | excluding hub member), |
| | | | | 74 | mm (total length, including |
| | | | | | hub member) |
| Calibrated | 0.85 | 0.65 | 0.10 | 65 | mm (needle shaft only, |
| Epidural | | | | | excluding hub member), |
| Needle | | | | 94 | mm (total length, including |
| | | | | | hub member) |
| Epidural | 0.85 | 0.75 | 0.05 | 700 | mm, excluding filter |
| Catheter | | | | 750 | mm, including filter |

8—22 Gauge:

| | O.D. | I.D. | Thickness | Length | |
|---|---|---|---|---|---|
| Sheath | 1.00 | 0.85 | 0.075 | 50 | mm (tubular member only, |
| | | | | | excluding hub member), |
| | | | | 69 | mm (total length including |
| | | | | | hub member) |
| Calibrated | 0.70 | 0.50 | 0.10 | 65 | mm (tubular member only, |
| Epidural | | | | | excluding hub member), |
| Needle | | | | 94 | mm (total length including |
| | | | | | hub member) |
| Epidural | 0.70 | 0.60 | 0.05 | 600 | mm, excluding filter |
| Catheter | | | | 650 | mm, including filter |

The above Tables provide dimensions for many of the sets between gauges 14-22, which are examples of sets embodying the invention.

In summary, embodiments of the invention may provide the following advantages:

1—No Accidental Disconnection at the Distal End of the Catheter.

This usually happens when the detachable Luer hub becomes loose. This may occur when mobilising the patient in the ICU and inside theatre, which may lead to contamination of the catheter, followed by possible infection of the skin, soft tissue, meningitis, or encephalitis. Thus, the modified system embodying the invention, which could be either of alternative constructions A and B, given below, has no Luer hub at all.

A—The distal end of the catheter is fused with the filter.

B—The distal end of the catheter is fused with a hub screwed internally, and can be fitted tightly to the filter, without causing obstruction to the flow of the infusion. This gives a-chance to change the filter in patients on long term epidural infusion.

2—There Will be no Occlusion of the Catheter Lumen by Over Tightening the Detachable Luer Hub, as the latter is no part of the modified system.

More particularly, when using an epidural apparatus in accordance with the invention, a sheath is employed instead of an epidural needle for guiding the catheter into and out of the body. By this time, the needle has been removed and disposed of. Accordingly, unlike conventional epidural sets which require a removable connection (such as a Luer hub) between the catheter and the hub, to allow for removal of the needle, when using an epidural apparatus in accordance with the invention, such a removable connection is no longer necessary and can be replaced by a fixed, preadjusted connection such as those of alternatives A and B above, which allows a predetermined setting of the desired flow rate, thus avoiding the above mentioned difficulties associated with over tightening or loosening of the connection.

3—Easy Method to Identify and to Confirm Location in the Epidural Space.

By rotating clockwise an epidural sheath having a tubular member of flexible plastics material and finding there is no resistance, means the needle is in the epidural space. If there is resistance on rotation, this suggests the epidural needle is not in the epidural space and requires insertion.

4—Less Incidence of Dural Puncture.

The process of slowly advancing the epidural needle through the ligamentum flavum, by rotating the needle inside the epidural sheath, allows very fine control of the movement of the needle and therefore minimises the accidental perforation of the dura (“dural tap”). The shorter bevel in the new epidural needle also helps to reduce this complication.

5—Increased Diameter of the Catheter

Increasing the internal diameter of the catheter increases the rate of flow of local anaesthetics because of a decrease in resistance inside the catheter, according to the well-known Hagen-Poiseuille equation.

6—Developing a Paediatric Epidural Set.

The modified set with its' increased catheter diameter, allows the development of a paediatric epidural set, using a small gauge needle.

7—Adjustments in Catheter Length

Adjustments can be made in the catheter length, through the epidural sheath, without fear of damaging the catheter.

8—Less Obstruction to Threading the Catheter in the Epidural Space.

There is a possibility of partial obstruction by the ligamentum flavum, if the mouth of the Touhy needle is partially in the epidural space and partially in the ligamentum flavum.

This is unlikely to happen with this system because of the shorter bevel of the needle and the sheath can be easily advanced to the epidural space over the epidural needle.

9—Less Trauma to the Soft Tissues.

There is less damage to the nerves and blood vessels, with less haematoma formation. This system overcomes the trauma caused by the cutting end of the Tuohy needle during withdrawal of the needle, because it is passing through a sheath of plastics material.

1. Epidural apparatus comprising
(a) an epidural needle comprising a needle shaft having a tip to allow insertion of a distal end of the needle shaft through the skin of a human or animal body;
(b) a sheath comprising a tubular member of plastics material adapted to surround a length of the shaft of the epidural needle but terminate short of the tip thereof,
which tubular member, at a distal end thereof is also capable of penetration through the skin following insertion into the skin of the tip of the shaft of the epidural needle and which epidural needle is thereafter removable from the sheath; and
(c) a catheter capable of passage through the sheath into the human or animal body;
whereby after arranging the sheath around the epidural needle and inserting the tip of the needle shaft and then a distal end region of each of the needle shaft and the sheath into the human or animal body, the epidural needle is removable from the sheath and the catheter is capable of insertion into the human or animal body through the sheath, thereby avoiding damage by the epidural needle of the catheter during passage through the tip thereof on insertion, adjustment of the length of the catheter within the body or removal of the catheter from the body.

2. Epidural apparatus according to claim 1, wherein the sheath additionally comprises a generally cylindrical hub member secured to an axial end of the tubular member and coaxial therewith, the hub member having a hollow bore so as to allow access of the needle shaft therethrough and through the tubular member.

3. Epidural apparatus according to claim 1 or claim 2, wherein the epidural needle additionally comprises a generally cylindrical hub member secured to an axial end of the needle shaft and coaxial therewith, the hub member having a hollow bore so as to allow access therethrough and through the needle shaft.

4. Epidural apparatus according to any preceding claim, additionally comprising means adapted to secure the epidural needle and sheath to, or remove the epidural needle and sheath from, one another.

5. Epidural apparatus according to claim 4, where the said means are additionally adapted to provide controlled relative axial movement of the epidural needle and sheath, one relative to the other.

6. Epidural apparatus according to claim 5, wherein the means for removably securing together the epidural needle and the sheath are
(1) an internally screw threaded surface defining a bore of a cylindrical collar coaxially surrounding and fixed relative to one of the shaft of the epidural needle and the tubular member of the sheath at a respective proximal end region thereof; and
(2) an externally screw threaded surface of a hub member axially aligned with and fixed relative to the other of the shaft of the epidural needle and the tubular member of the sheath at a respective proximal end region thereof, which externally screw threaded surface is cooperable with the internally screw threaded surface of the collar to allow screw threaded engagement of the hub member within the bore of the collar.

7. Epidural apparatus according to claim 6, wherein the means for removably securing together the epidural needle and the sheath are
(1) an internally screw threaded surface defining a bore of a cylindrical collar surrounding and fixed relative to the shaft of the epidural needle and
(2) an externally screw threaded surface of a hub member of the sheath at a proximal end thereof and cooperable with the internally screw threaded surface of the epidural needle.

8. Epidural apparatus according to claim 6 or claim 7, wherein at least one of the epidural needle and the sheath has, surrounding and fixed relative thereto, a respective disc-like calibration member provided with marking so as to allow an indication of the extent of relative revolution one to another and thereby, from the pitch of the screw threads, allow calibration of the extent of relative axial movement.

9. Epidural apparatus according to claim 8, wherein the disc-like calibration member is provided on the tubular or hub member of the sheath.

10. Epidural apparatus according to any one of claims 2 to 9, wherein the sheath is an integral unit of plastics material and the tubular member projects axially from an axial end of the hub member.

11. Epidural apparatus according to any preceding claim, wherein the tubular member of the sheath is of flexible plastics material.

12. Epidural apparatus according to any one of claims 2 to 9, wherein the tubular member of the sheath fits tightly at a proximal end thereof within the distal end of the bore of the hub member of the sheath, the tubular member being of flexible plastics material and the hub member being of rigid plastics material.

13. Epidural apparatus according to any one of claims 2 to 12, wherein the bore of the hub member of the sheath has a diameter, at a distal end thereof, essentially equal to the internal diameter of the tubular member and, at a proximal end thereof, a wider diameter.

14. Epidural apparatus according to any preceding claim, wherein the epidural needle additionally comprises a generally cylindrical hub member secured to an axial end of the needle shaft and coaxial therewith, the hub member having a generally hollow bore so as to allow access through the epidural needle.

15. Epidural apparatus according to claim 14, wherein the bore of the hub member of the epidural needle has a diameter, at a distal end thereof, essentially equal to the internal diameter of the needle shaft and, at a proximal end thereof, a diameter sufficiently wide to receive a distal end region of a syringe.

16. Epidural apparatus according to claim 14, as appendent to claim 6, wherein the collar projects axially from a distal end of the hub member.

17. Epidural apparatus according to claim 8, wherein each of a hub member of the epidural needle and a tubular or hub member of the sheath carries in fixed relation with and coaxial with the epidural needle and sheath respectively, a respective disc-like core element having a toothed outer circumferential surface and wherein the epidural apparatus additionally comprises an annular disc-like outer element having a marked outer circumferential surface and a toothed inner circumferential surface cooperable, on axial movement thereof along one of the epidural needle and the sheath, with either of the respective core elements upon engagement therewith so as to provide the said disc-like calibration member on one of the sheath and epidural needle as desired.

18. Epidural apparatus according to claim 1, wherein the epidural needle has a body part which is a one-piece moulding of plastics material having a generally hollow bore passing axially therethrough, the body part comprising, (1) at a proximal end region, a hub part capable of receiving within the bore the hub of a syringe and capable of being held externally by the user, (2) at a distal end region, a cylindrical collar projecting axially away from a distal end of the hub member and having an internally screw threaded bore adapted to receive an externally screw threaded sheath and, (3) in a medial axial region of the body part, a disc-like core element provided by a portion of greater radial extent than the hub member and having a toothed outer circumferential surface adapted to receive an annular disc-like outer element having correspondingly toothed inner circumferential surface and a marked outer surface.

19. Epidural apparatus according to any preceding claim, which additionally includes
(1) a stylet comprising an essentially cylindrical solid rod adapted to pass into the epidural needle up to the tip of the needle shaft and thereby block the distal end of the needle shaft during insertion thereof into a human or animal body, thereby preventing egress of matter into the needle shaft; and
(2) means adapted to removably secure the epidural needle and stylet to one another.

20. Epidural apparatus according to claim 19, wherein the means for removably securing together the stylet and the epidural needle are
(1) an internally screw threaded surface defining a bore of a cylindrical collar coaxially surrounding and fixed relative to one of the stylet and the epidural needle at a respective proximal axial end region thereof; and
(2) an externally screw threaded surface of a hub member axially aligned with and fixed relative to the other of the stylet and the epidural needle, which externally screw threaded surface is cooperable with the internally screw threaded surface of the collar to allow screw threaded engagement of the hub member within the bore of the collar.

21. Epidural apparatus according to claim 20, wherein the means for removably securing together the stylet and the epidural needle are
(1) an internally screw threaded surface defining a bore of a cylindrical collar surrounding and fixed relative to a proximal end region of the stylet and
(2) an externally screw threaded surface of a hub member of the epidural needle at a proximal end region thereof and cooperable with the internally screw threaded surface of the collar.

22. Epidural apparatus according to any preceding claim, wherein an axial end of the catheter is fused with a filter.

23. Epidural apparatus according to any preceding claim, wherein an axial end of the catheter is fused with a hub provided on a filter.

24. A method of locating a catheter within an epidural space of a human or animal, which method comprises
(1) providing (a) an epidural needle comprising a needle shaft having a tip to allow insertion of a distal end of the needle shaft through the skin of a human or animal body and (b) a sheath comprising a tubular member of plastics material adapted to surround a length of the shaft of the epidural needle but terminate short of the tip thereof and capable of penetration through the skin following insertion into the skin;
(2) inserting into the skin a distal end region of each of the shaft of the epidural needle and the tubular member of the sheath until the tip of the needle shaft reaches the epidural space;
(3) thereafter removing the epidural needle from the sheath to leave only the distal end region of the tubular member of the sheath within the body; and thereafter
(4) inserting the catheter into the epidural space by passage thereof through the sheath.

25. A method according to claim 24, wherein the tubular member is of flexible plastics material and wherein means are provided for controlling relative axial movement of the epidural needle and sheath one relative to the other, and wherein step (2) is carried out by
(i) arranging the tubular member of the sheath around the shaft of the epidural needle at a position where the distal end of the tubular member terminates short of the tip of the shaft;
(ii) inserting the tip of the shaft of the epidural needle and a distal end region of the tubular member into the body to a position no further than the ligamentum flavum;
(iii) advancing the distal end of the tubular member axially relative to the shaft until resistance to the movement is sensed;
(iv) advancing the tip of the needle shaft through a predetermined minimal axial extent relative to the distal end of the tubular member; and
(v) again advancing the distal end of the tubular member axially forward of the needle shaft and sensing the resistance thereto;
(vi) repeating steps (iv) and (v) until no resistance is sensed, thus locating the tip of the epidural needle in the epidural space; and
(vii) further advancing the flexible tubular member axially forward of the needle shaft into the epidural space.
